(19) United States
(12) Patent Application Publication
(10) Pub. No.: US 2006/0258909 A1
(43) Pub. Date: Nov. 16, 2006

(54) Methods and apparatus for maintaining sterility during transluminal procedures

(75) Inventors: Vahid Saadat, Saratoga, CA (US); John A. Cox, Rancho Santa Margarita, CA (US); Tracy D. Maahs, Rancho Santa Margarita, CA (US)

Correspondence Address: LEVINE BAGADE HAN LLP, 2483 EAST BAYSHORE ROAD, SUITE 100, PALO ALTO, CA, 94303, US

(73) Assignee: USGI Medical, Inc., San Clemente, CA (US)

(21) Appl. No.: 11/400,297

(22) Filed: Apr. 7, 2006

Related U.S. Application Data
(60) Provisional application No. 60/669,412, filed on Apr. 8, 2005.

(51) Int. Cl. A61B 1/00

(52) U.S. Cl. 600/121

(57) ABSTRACT

Methods and apparatus for maintaining sterility during transluminal procedures are described. The sterility is achieved by making use of a membrane in either the form, e.g., of a sheath, cap or umbrella to keep the instruments (endoscopes, overtubes, etc.) sterile while being advanced through lumens in the body and into the various cavities. Methods, such as vacuum acquisition, mechanical tissue anchoring, etc., of the underlying tissue followed by incision of the tissue with, e.g., a needle knife, can be utilized to gain access to the various cavities. It is expected that this procedure will provide the desired level of sterility for transluminal procedures.

### CROSS-REFERENCE TO RELATED APPLICATIONS

This application claims the benefit of priority to U.S. Prov. Pat. App. Ser. No. 60/669,412 filed Apr. 8, 2005, which is incorporated herein by reference in its entirety.

### BACKGROUND OF THE INVENTION

The present invention relates to methods and apparatus for carrying out transluminal procedures within a human body. More particularly, methods and apparatus are described for maintaining the sterility of tools and devices delivered through apparatus such as endoscopes, overtubes (rigidizable or otherwise), etc., during transluminal procedures.

As endoluminal instruments are inserted into a patient body through a natural orifice, such as a patient's mouth or anus, bacteria and other foreign bodies may be carried by the inserted endoluminal instrument and seeded or transferred to other regions within the body. For instance, an endoluminal instrument may be inserted through a patient's mouth, where bacteria may be picked up by the instrument and carried through the patient's esophagus and into the stomach. As the instrument is advanced, e.g., transgastrically into the peritoneal space of the patient, the bacteria may be transferred by the instrument to tissues within the peritoneal space possibly resulting in an intra-abdominal infection.

One conventional procedure for maintaining sterility during, for instance, a transgastric procedure where the instrument may be advanced into a patient's mouth, through the esophagus and into the stomach, and then across the wall of the stomach or intestinal tract, typically involves repeatedly flushing the oral and stomach cavities with a sterile solution. This technique, however, is insufficient to maintain the standard of sterility required for such procedures, for example, especially when the stomach wall is pierced and a device is advanced therethrough and into the peritoneal cavity.

### BRIEF SUMMARY OF THE INVENTION

The following describes several techniques for the sterilization of some common transluminal procedures and surgeries that involve the use of an endoscope or overtube. The diagrams provided are illustrations of the same.

One solution suggested for maintaining the sterility of the instrument is described as follows. The surgeon, who is within a sterile field relative to a patient, operates the instruments from behind a closed and sterile screen/curtain, in order to avoid contact with any of the contaminants that may affect the procedure. All operations involving the instruments may be handled from the side of the curtain on which the surgeon operates from.

In one variation, a protective and sterile membrane is placed at or over the end of the endoscope or overtube (rigidizable or otherwise), the inner lumen or lumens of which have been sterilized beforehand. This membrane helps to maintain the sterility of the interior of the endoscope or overtube and the instruments that may be passed through it when the endoscope or overtube is advanced into or through a patient body. This membrane is designed in such a way, using the appropriate materials such that it can be ruptured easily, once the instrument is secured at or adjacent to a tissue region of interest.

Examples of rigidizable overtubes which may be utilized with the methods and apparatus described herein and which are configured to transition between a flexible shape during advancement within a patient and also a rigidized state where the device may be rigidized to maintain a selected configuration may be seen in further detail in U.S. Pat. Nos. 6,783,491; 6,790,173; 6,837,847; and 5,251,611, each of which are incorporated herein by reference in its entirety. Other examples of rigidizable and steerable multi-lumen devices are shown and described in further detail in U.S. patent application Ser. No. 10/346,709 filed Jan. 15, 2003; Ser. No. 10/458,060 filed Jun. 9, 2003; and Ser. No. 10/797,485 filed Mar. 9, 2004, each of which is incorporated herein by reference in its entirety.

This procedure ensures the sterility of any instruments which may be advanced through the endoscope or overtube positioned within the patient prior to advancing these instruments, e.g., through the stomach wall and into the peritoneal cavity. The membrane can be ruptured at a specific location where the instruments are advanced out of the covered endoscope or overtube with the help of a variety of devices. Once the membrane is ruptured and the pathway cleared, the instruments can be advanced out of the endoscope or overtube.

A method such as vacuum acquisition can be utilized to draw a section of the stomach wall into or against the overtube, and the adhered stomach tissue may then be incised with, e.g., a needle knife, a scalpel, an energizable probe, etc., to provide access to the peritoneal cavity. This procedure can be applied to any of the variations described herein.

Another alternative to the protective membrane is a sterile sheath that envelopes a portion of or the entire endoscope or overtube. The endoscope or overtube may be inserted into the patient with this sheath covering the endoscope or overtube. Moreover, this sheath may be configured to be easily penetrated at specific locations, e.g., at its distal end, and the instruments within the endoscope or overtube may be kept sterile while contained within the sheath.

In yet another variation, an expandable sheath is attached to or at a location proximal to the distal end of the endoscope or overtube. The expandable sheath may be maintained in a low-profile configuration during advancement through the patient body and can be deployed through a variety of methods, e.g., expanding nitinol wires, similar to a conventional umbrella, spring stainless steel, pull wires, etc. This expandable sheath may also be configured into a low-profile delivery configuration and delivered through the endoscope or overtube itself.

Further transluminal procedures involving the use of the devices mentioned herein are also described herein. Examples of various transgastric or transluminal procedures may be seen in further detail in U.S. patent application Ser. No. 10/918,217 filed Aug. 11, 2004, which is incorporated herein by reference in its entirety.

### BRIEF DESCRIPTION OF THE DRAWINGS

FIG. 1A is a schematic diagram of a conventional endoscope or rigidizable overtube.

FIG. 1B is a schematic diagram of a sterile sheath that may be used to cover the endoscope or overtube.

FIG. 2A represents one configuration of the sheath before being deployed onto the endoscope or overtube.

FIG. 2B is the plan view of the same.

FIG. 2C is an alternative configuration of the sheath in FIG. 1B.

FIG. 3 is a view representing how the sheath may be loaded onto the endoscope or overtube.

FIG. 4 is a perspective view of how the entire system may be used on a patient.

FIGS. 5A-5B are views of a cap loaded with the sterile membrane at the distal end.

FIGS. 6A-6B represent how the cap in FIGS. 5A and 5B may be loaded onto the endoscope or overtube.

FIG. 6C illustrates how the variation of the device shown in FIGS. 6A-6B may be advanced into the stomach of a patient through the esophagus.

FIGS. 6D-6E illustrate how the distal end of the endoscope or overtube may be attached to the stomach wall and a section of it drawn into the tube using a vacuum; an incision may be made by, e.g., a needle knife, advanced through one of the working channels of the overtube.

FIG. 7 is a perspective view of how the alternative system may be used on a patient.

FIGS. 8A-8B illustrates how the endoscope or overtube can tear through the sterile sheath.

FIGS. 9A-9B illustrates the process of advancing the overtube enveloped by the sheath into the stomach through the esophagus.

FIG. 9C is a perspective view of how the entire system may extend through the gastrointestinal system of a patient.

FIGS. 10A-10B are views of the pre- and post-deployed configurations of an alternative variation of the overtube.

FIG. 11 is a perspective view illustrating use of the variation on a patient.

FIG. 12A is a close up of the system in FIG. 11 showing the tissue engaging elements.

FIGS. 12B-12C illustrate perspective and end views, respectively, of an alternative variation of the device of FIG. 12A having a circumferential outer lip or flange which may be engaged temporarily to a tissue surface.

FIGS. 12D and 12E show side views of alternative methods for temporarily engaging the sheath to the tissue surface.

FIGS. 12F-12H illustrate perspective and side views of another variation of the device having a circumferential inner lip or flange.

FIGS. 13A-13B illustrate alternative methods of attaching the endoscopic device or overtube to the tissue.

FIG. 14 shows a method of advancing an endoscopic device into the stomach where one or both of the instruments advanced may be rigidized or remain flexible.

FIG. 15 illustrates the pre-directability of the endoscopic device within the stomach. The device may be advanced into different areas of the stomach depending on the objective.

FIGS. 16A, 16B and 16C illustrate the process of advancing the device transvaginally and through the posterior cul-de-sac for access into the peritoneal cavity.

FIGS. 17A, 17B and 17C illustrate the process of advancing the device transcolonically to access the peritoneal cavity.

FIGS. 18A, 18B and 18C illustrate the process of advancing the device transthoracically, through the lungs into the thoracic cavity.

FIGS. 19A, 19B and 19 C illustrate the process of advancing the device through the esophagus for access into the thoracic cavity.

FIGS. 20A, 20B and 20C illustrate the process of advancing the device transvesically for another variation of access into the peritoneal cavity.

### DETAILED DESCRIPTION OF THE INVENTION

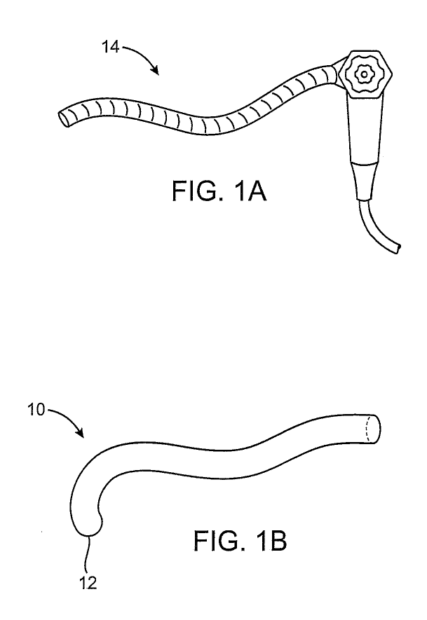

Referring to FIGS. 1A-1B, a variation of the apparatus is described. The sheath 10 shown in FIG. 1B may be made of an elastic material (for example, plastic) that can be ruptured easily at its distal end 12. Alternatively, the distal end 12 may be configured as an overlapped portion or as a valve-type opening which may be opened simply via an instrument being pushed gently or otherwise against the distal end 12. The distal end 12 is described in further detail below. The sheath may fit loosely over the endoscope or overtube 14 or it may be optionally configured to have an expanded diameter which is sufficiently smaller relative to the endoscope or overtube 14 diameter such that the sheath 10 clings to the endoscope or overtube 14 in such a way as to not disturb the insertion of the endoscope or overtube 14 into the patient.

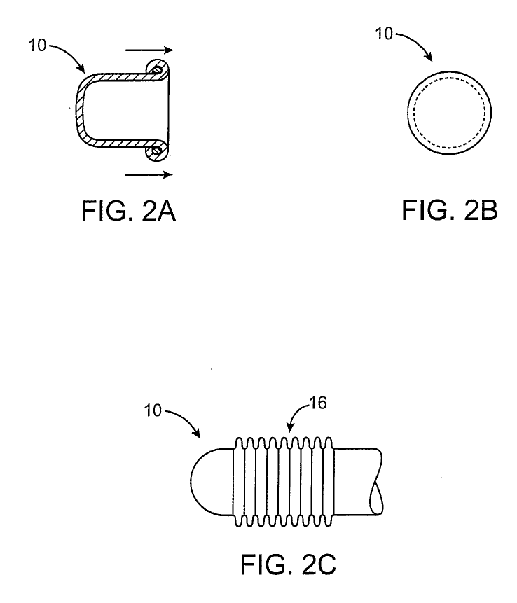

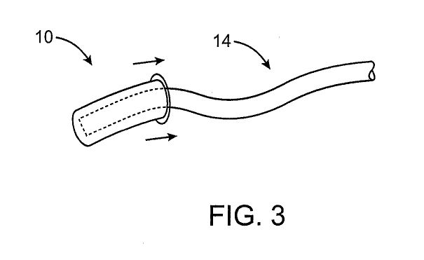

FIGS. 2A and 3 illustrate examples of how the sheath 10 can be rolled up or everted upon itself and then rolled or loaded onto the endoscope 14. FIG. 2A shows sheath 10 alone for clarity and illustrates how the rolled up sheath 10 may be simply unrolled for positioning over endoscope or overtube 14. FIG. 2B shows a plan view of the rolled sheath 10. In another variation of the sheath, a length of the sheath 10 may be everted or intussuscepted 16 upon itself to collapse into a compact configuration. Once the endoscope or overtube 14 is to be covered, intussuscepted sheath 16 may be simply slid onto the device unraveling itself over an outer surface of the endoscope or overtube 14. Once this sheath 10 is loaded onto the endoscope or overtube 14, the endoscope or overtube 14 can be inserted into the patient per-orally without fear of contamination of the instruments therein.

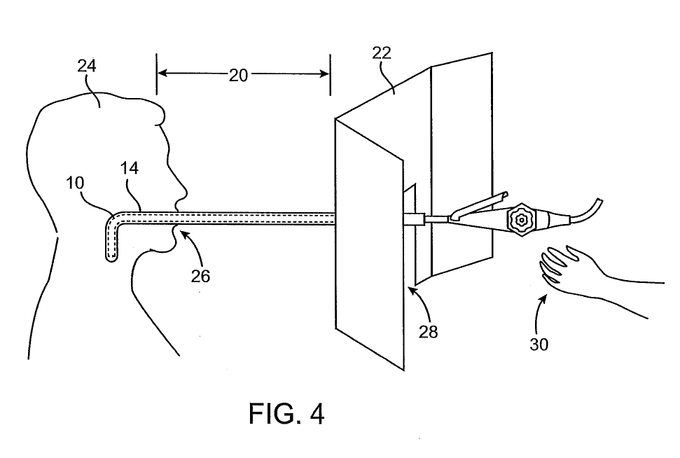

FIG. 4 shows a schematic illustration of one example of how sheath 10 may be utilized during a procedure to maintain sterility of the instruments through the patient body. Once sheath 10 has been loaded onto endoscope or overtube 14, the device may be passed through an opening 28 defined in a sterile dressing or curtain 22, behind which an operator 30 may be positioned. The sterile dressing or curtain 22 may be placed directly upon the patient 24 or held as a barrier between the operator 30 and patient 24. In either case, passing the instrument through the sterile dressing or curtain 22 and into the patient's mouth 26 may extend the sterile field 20 from dressing or curtain 22 to the patient's mouth 26.

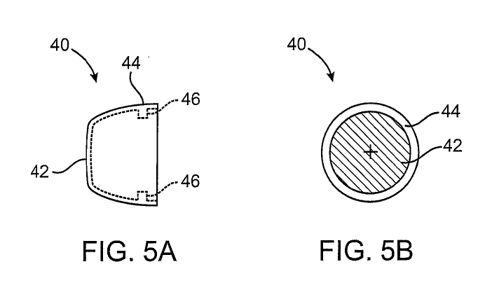

With reference to FIGS. 5A-5B, another variation 40 of the sterile sheath is described. As shown, a sterile membrane 42 may be loaded or held on the end of a circumferential cap or hood 44, which may be attached to the distal end of the endoscope or overtube 14 via a retaining mechanism 46 such as a detent or projection. This membrane 42 may be made of an elastic, plastic, or woven material that can be easily breached. Since the interior of the endoscope or overtube 14 is covered by sterile sheath 40 and is not exposed to the environment within the patient body, particularly the mouth, until the membrane 42 is breached, the sterility can be maintained within the endoscope or overtube 14.

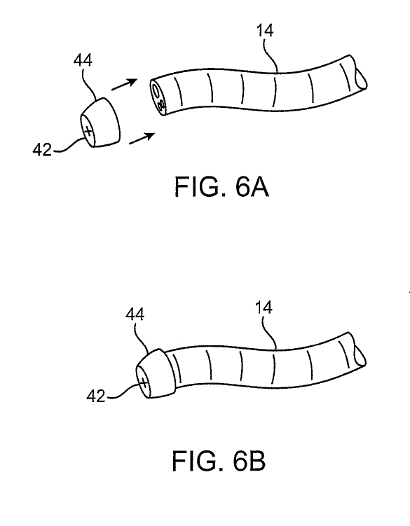

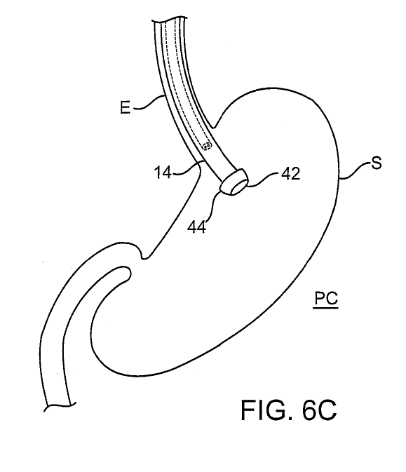

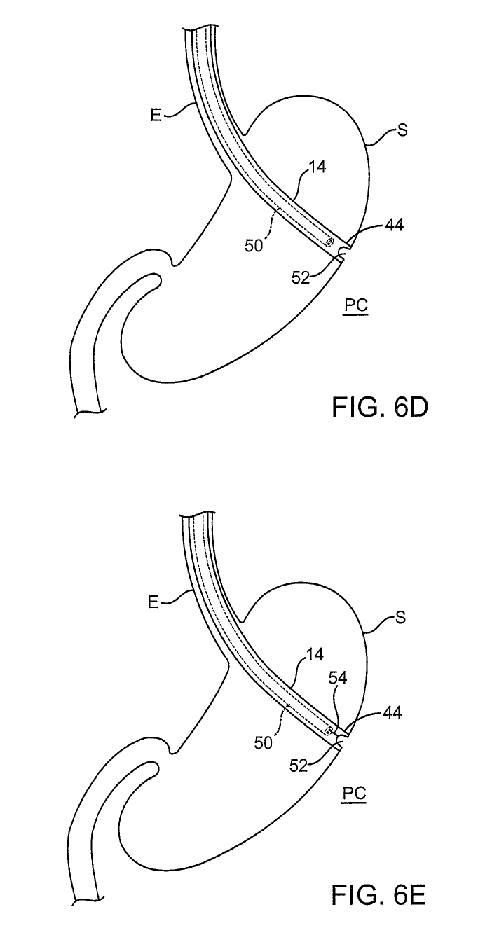

FIGS. 6A-6B illustrate how the cap or hood 44 can be attached directly onto the distal end of the endoscope or overtube 14. The subsequent FIGS. 6C, 6D, and 6E illustrate an example of advancing the rigidizable overtube 14 having sterile cap or hood 44 disposed on a distal end of the device. As the overtube 14 is advanced, as shown in FIG. 6C, through the patient's esophagus E and into the stomach S, the distal end of overtube 14 may be directed to a region of stomach tissue through which a gastrotomy is to be made for entry into the peritoneal cavity PC. Once the distal end of the overtube 14 and its sterile cap or hood 44 has been directed against the interior mucosal lining of the interior stomach, a separate endoscope 50 and/or piercing instrument 54, such as a needle knife, energizable probe, etc., may be advanced through a lumen of the overtube 14, as shown in FIG. 6D. The instrument 54 may then be actuated or manipulated to pierce through the sterile barrier 42 and end cap 44 and into the stomach tissue, as shown in FIG. 6E. Because the interior of the overtube 14 has not been breached during advancement into the stomach and prior to piercing through membrane 42, the instruments delivered through overtube 14 may maintain its sterility.

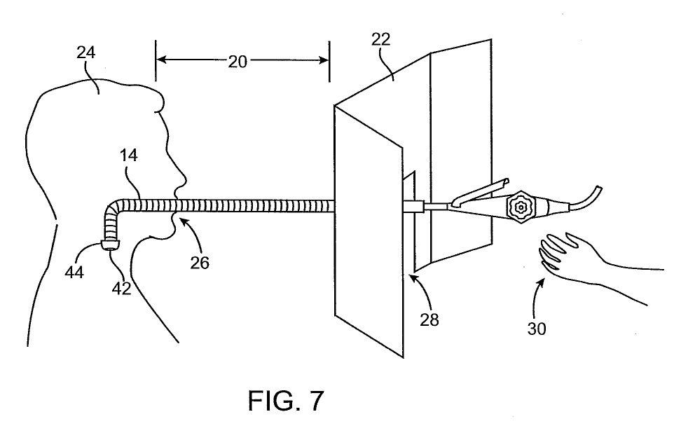

To facilitate piercing through the stomach tissue and entry into the peritoneal cavity PC, vacuum suction may be applied against the mucosal lining of the stomach S to draw a section 52 of the stomach wall into the overtube 14 or at least against the overtube 14 prior to making the incision through the tissue. This incision provides access to the peritoneal cavity PC of the patient. FIG. 7 shows an illustration of one possible arrangement and deployment of this variation which is similar to that shown and described above in FIG. 4 with the addition of cap or hood 44 positioned upon the distal end of the endoscope or overtube 14.

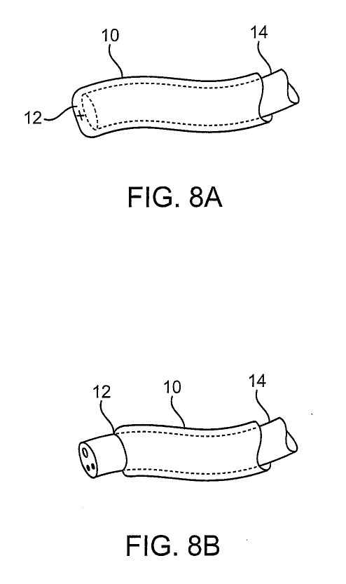

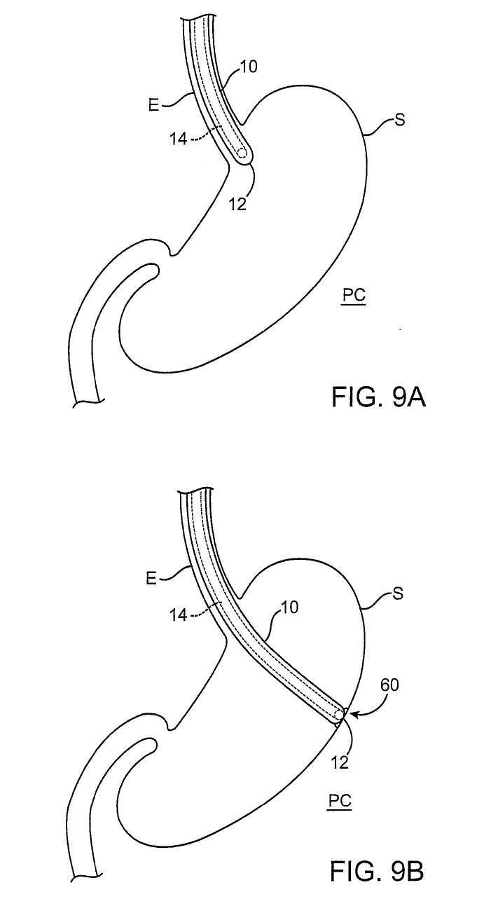

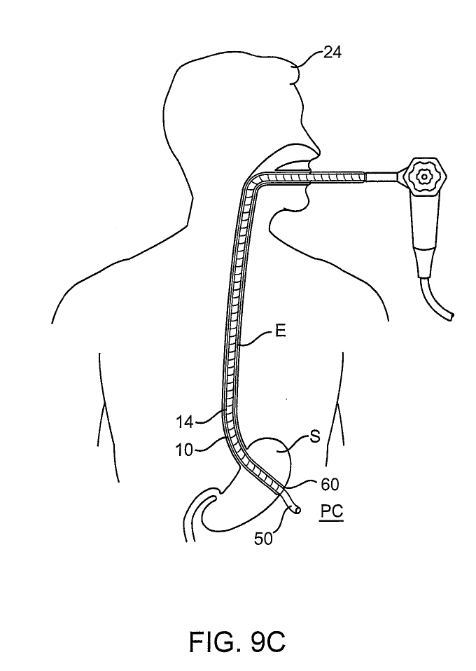

With reference to FIG. 8A-8B, the distal end 12 of sterile sheath 10 can be made to be breached or opened by endoscope or overtube 14 when urged distally relative to the sheath 10. As such, distal end 12 may be configured to be perforated or may be formed as having a relatively thinner section than the rest of sheath 10 such that pressure brought to bear against distal end 12 will rupture the integrity of the sheath material consistently at the distal end 12 rather than at an unintended location. Alternatively, as mentioned above, the distal end 12 may be configured as an overlapped portion or as a valve-type opening which may be opened simply via an instrument being pushed gently or otherwise against the distal end 12. In use, overtube 14 may be advanced trans-esophageally through esophagus E and into the stomach S with sterile sheath 10 disposed thereupon, as shown in FIG. 9A. Sheath 10 may be seen as extending over the entire length of overtube 14 and out of the mouth of the patient 24 to create a sterile channel through sheath 10 extending from outside the patient to the point of entry through the stomach wall. Once the distal end of overtube 14 has been brought into proximity with the desired region of stomach tissue, as shown in FIG. 9B, a cutting instrument may be advanced through overtube 14, which is sterile, and pierced or otherwise urged through the distal end 12 of sheath 10 and through the stomach wall, thereby creating a gastrotomy 60 into the peritoneal cavity PC. As shown in FIG. 9C, once the distal end 12 has been pierced or ruptured and the gastrotomy 60 created, an endoscope 50 or other instrument may be passed through the sterile channel of the sheath 10 and into the peritoneal cavity. The ruptured distal end 12 may also be fabricated to retain some stiffness such that when the overtube 14 is retracted from or withdrawn through the ruptured distal end 12, the opening may remain in an open configuration.

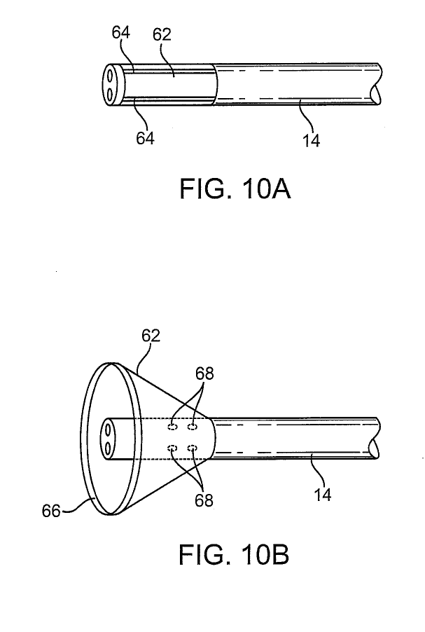

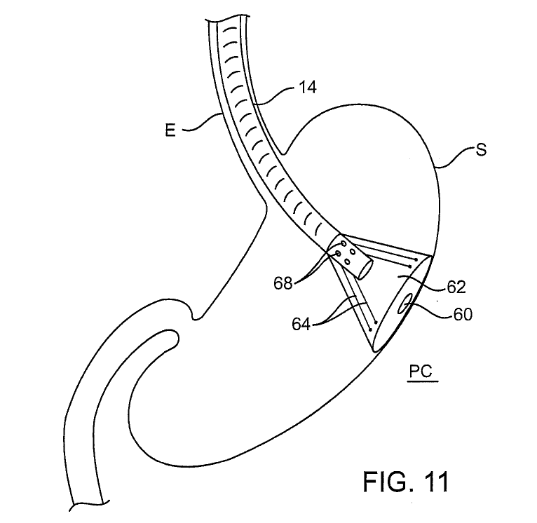

Referring now to FIGS. 10A-10B, another variation of the apparatus is shown and described which is configured to be advanced into the patient stomach and placed against an interior wall for sterilizing the tissue region of interest prior to creating a gastrotomy therethrough to access the peritoneal cavity. This variation generally comprises an elastic sheath 62 loaded or attached near or at the distal end of the endoscope or overtube 14. A number of Nitinol wires or struts 64 may provide structural support to the collapsible sheath 62 and further aid the deployment of the elastic sheath 62 from the collapsed configuration, as shown in FIG. 10A, into an expanded configuration, as shown in FIG. 10B. The Nitinol wires or struts 64 may be attached to the sheath 62, much like the wires in an umbrella, although other methods for attachment as known in the art, may also be utilized. Once the endoscope or overtube 14 advances into the stomach S, as shown in FIG. 11, the wires 64 may be released or otherwise urged to deploy. Although Nitinol wires are described, other examples may also utilize other methods or materials such as spring stainless steel, etc. Once sheath 62 has been deployed, a conformable edge or lip of sheath 62 may be placed into contact against the stomach tissue surface and a sterile fluid such as saline, water, iodine, or some other disinfecting agent may be infused through ports 68, defined along endoscope or overtube 14 within sheath 62, to clean and sterilize the stomach region. Once the tissue area has been sufficiently cleaned, any number of instruments may be advanced through endoscope or overtube 14 to create a gastrotomy 60 through the stomach wall.

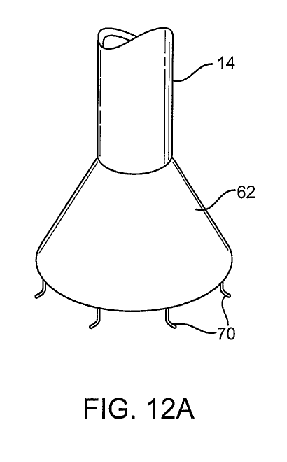

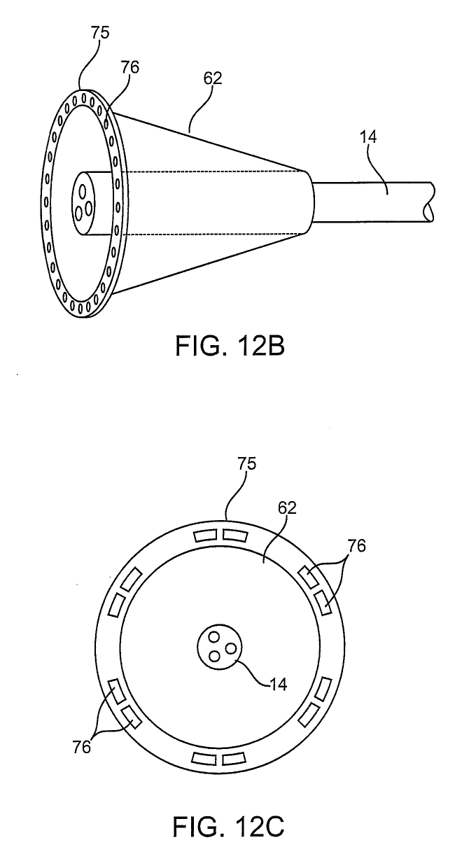

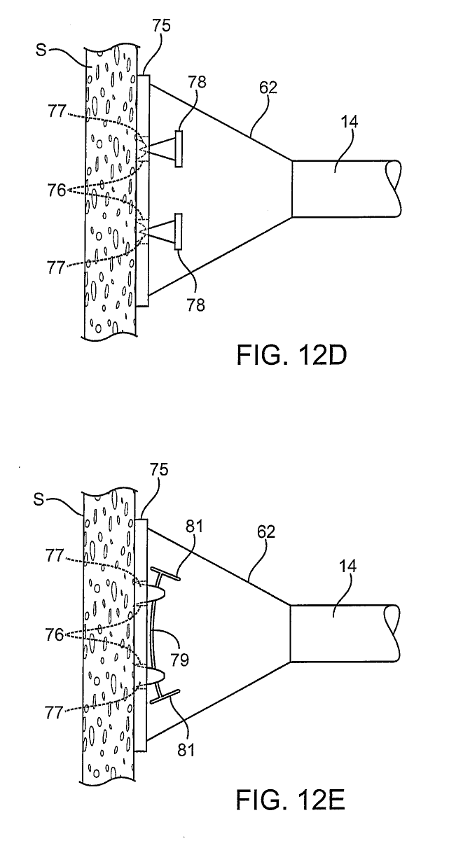

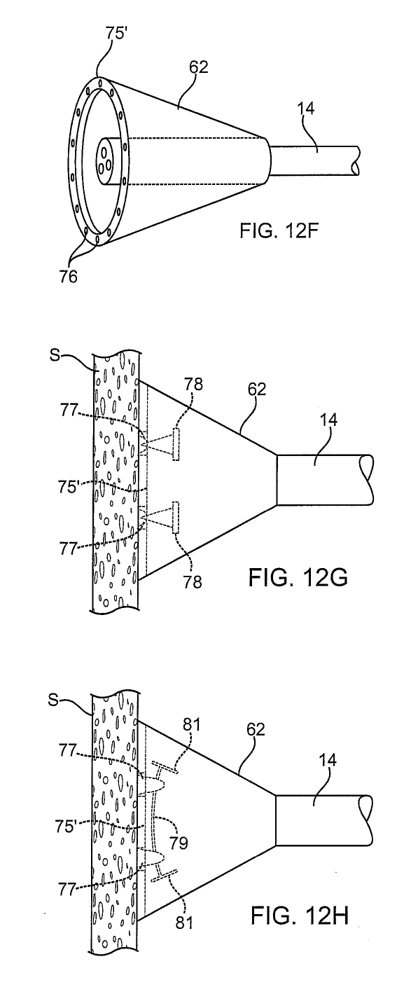

FIG. 12A illustrates that one or more anchors or barbs 70 may be placed at end of the sheath 62 to attach the device to the inner wall of the stomach prior to creating the gastrotomy. FIGS. 12B and 12C show perspective and end views, respectively, of a variation of the device of FIG. 12A in which sheath 62 may have a circumferential outer lip or flange 75 which is radially configured around the edge of sheath 62. Outer lip or flange 75 may define one or more features 76 around the flange 75 which provide for an engagement point or points with the underlying tissue. For instance, feature 76 may be defined as openings or eyelets through which tissue engaging mechanisms may be employed such as sutures, clips, staples, biocompatible adhesives, etc. Feature 76 may also be configured alternatively as projections or slots around or through which tissue engaging mechanisms may be passed to temporarily secure sheath 76 against the tissue surface.

An example is illustrated in the side view of FIG. 12D, which shows sheath 62 temporarily adhered to the stomach wall S via clips 78 (e.g., endoscopic hemostasis clips available from Olympus America, Melville, N.Y.), which may be passed through one or more features 76 and onto the tissue surface. Alternatively, portions of tissue 77 may be pulled through one or more openings 76 and temporarily secured against outer lip or flange 75 via a length of suture 79 which is secured via one or tissue anchors 81, as shown in FIG. 12E. Clips 78 and anchors 81 may be deployed against the tissue and flange 75 by another endoscopic instrument passed adjacent to endoscope or overtube 14 and removed by simply cutting suture 79 or releasing clips 78 and removing them from the region once flange 75 is to be released.

FIGS. 12F-12H show perspective and side views, respectively, of another variation in which sheath 62 may define a circumferential inner lip or flange 75′ having one or more features 76 defined thereon, as above. However, inner lip or flange 75′ is defined radially inward within sheath 62. In this variation, clips 78 or suture 79 may be similarly deployed through the tissue 77, as above, from within sheath 62.

In adhering the distal end of the elongate endoscope or overtube 14 to the interior of the stomach wall, various attachment mechanisms may be utilized. Examples are shown in FIGS. 13A and 13B describe possible variations of methods for attaching the endoscope or overtube 14 against the stomach interior, as shown in FIGS. 6C to 6E. The variation shown in FIG. 13A illustrates one or more helical corkscrews 74 rotatably positioned around the distal edge 72 of endoscope or overtube 14. FIG. 13B shows another variation in which one or more barbed anchors 74 may be positioned around the distal edge 72 for adherence to the stomach wall. These variations may be made use of in any of the following examples of transluminal procedures within the human body.

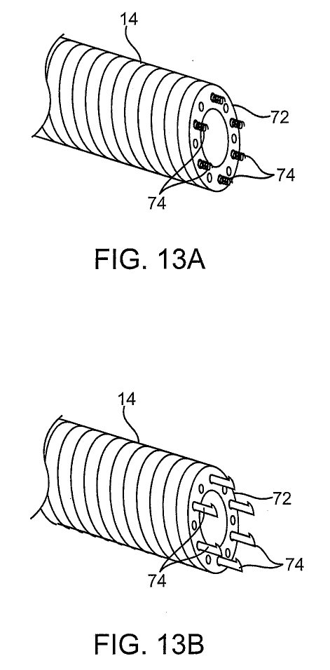

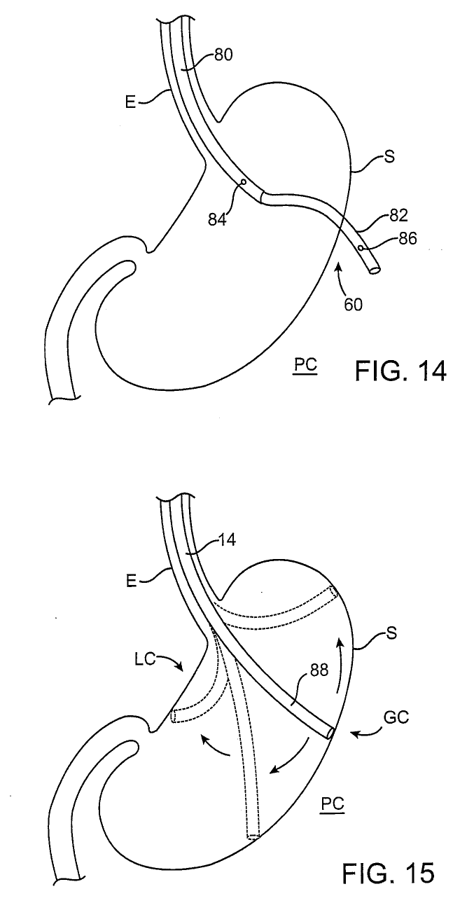

FIG. 14 shows yet another instrument and method of advancing an endoscopic device into the stomach S where either the elongate instrument may be comprised of at least a first section 80 and a second section 82, distal to the first section 80. One or both sections 80, 82 may be independently transitionable between a flexible and rigid state and one or both sections may additionally have ports 84, 86 defined along their outer surface for vacuum acquisition of tissue or for the delivery of fluids therethrough.

With reference to FIG. 15, the endoscope or overtube 14 is not restricted to access only one side of the peritoneal cavity PC. The steering of at least the distal end or distal portion 88 of the endoscopic device or overtube 14 allows the distal end 88 to be directed to any area within the stomach S. The distal end 88 may be steered and/or attached to a pre-determined region within the stomach closest to a region within the peritoneal cavity PC where a procedure is to be performed. For instance, the distal end 88 may be attached along the stomach at a region of the stomach antrum along the greater curvature GC for directly accessing the colon within the peritoneal cavity PC. In another example, the distal end 88 may be steered and attached to the stomach wall along the lesser curvature LC for directly accessing anatomical structures such as the pancreas, gall bladder, etc.

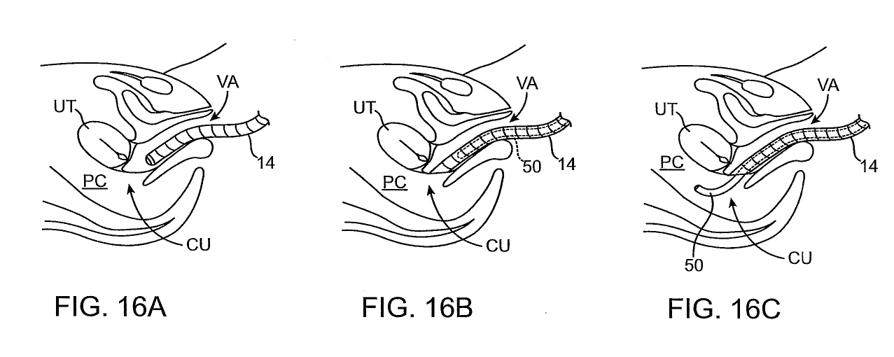

The instruments and methods described above may be utilized in any number of transluminal procedures aside from transgastric access. One example for a transluminal procedure is shown in FIGS. 16A to 16C where overtube 14 having a sheath 10 or sterile cap 40 may be advanced into a female patient's vagina VA and advanced trans-vaginally towards the uterus UT to obtain access to the peritoneal cavity PC. Access may be obtained by passing through (or passing tools through) an incision near or at the posterior cul-de-sac CU and advancing the instruments 50 therewithin. This technique may have applications for procedures such as tubal ligation, removal of the fallopian tube or tubes, transvaginal hysterectomies, etc.

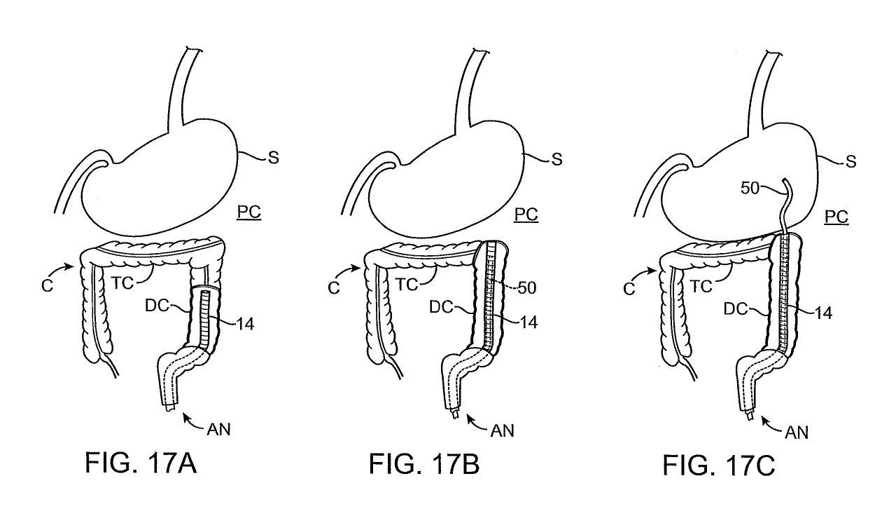

Another transluminal procedure is shown in FIGS. 17A to 17C in which overtube 14 may be inserted through a patient's anus AN and advanced transcolonically through the descending colon DC into the body. Overtube 14 may be advanced to a region within the colon C, such as the transverse colon TC, where the area may be sterilized in any manner as described above and endoscope 50 may be advanced through the colon wall and into the peritoneal cavity PC. Surgical procedures and other operations within the peritoneal cavity PC may then be performed therewithin as described above, e.g. on the outer wall of the stomach S.

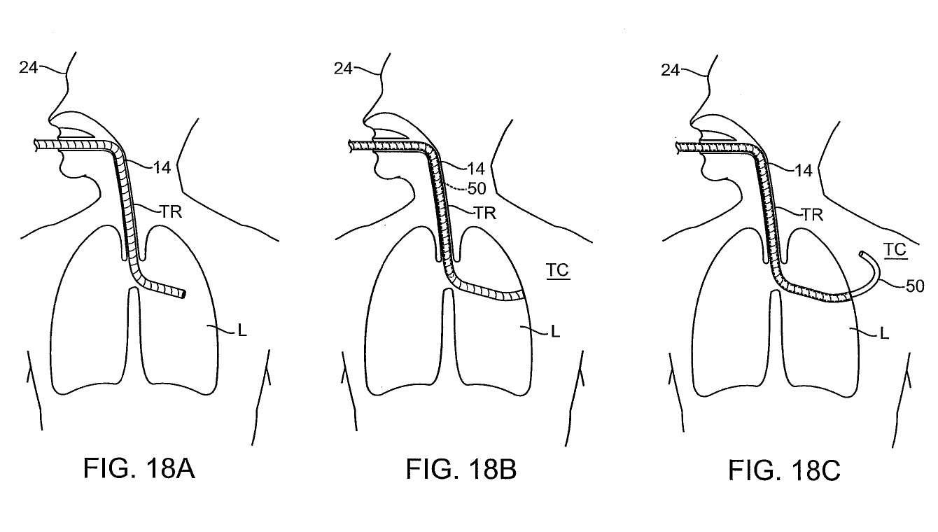

Yet another transluminal procedure is shown in FIGS. 18A to 18C in which overtube 14 may be advanced per-orally and through the trachea TR for access to the lungs L. Access may also be gained in this way to the thoracic cavity TC for carrying out, e.g., procedures upon the heart, diaphragm, etc. Alternatively, the overtube 14 may be advanced into the lungs L without gaining access to the thoracic cavity TC. Thus, procedures may be carried out entirely within the lungs L.

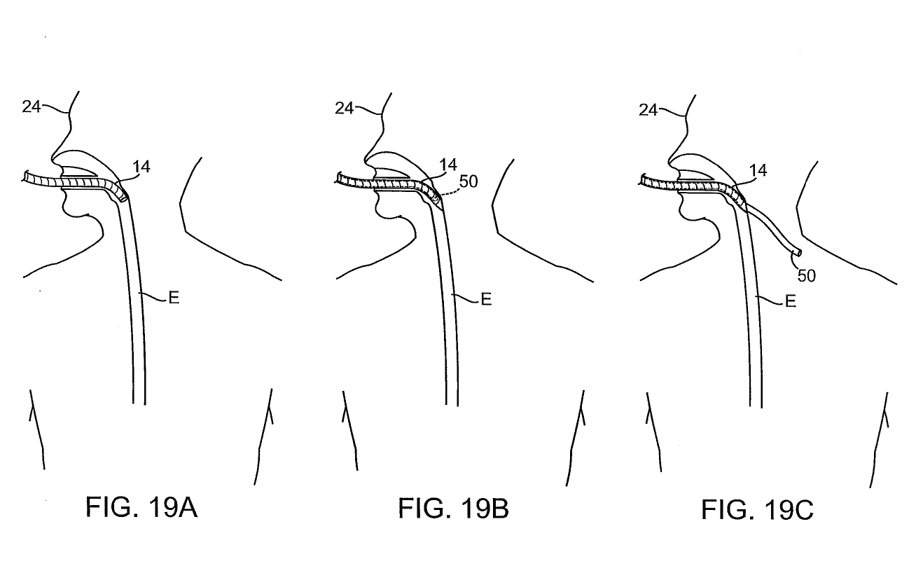

Another transluminal procedure is shown with reference to FIGS. 19A to 19C where overtube 14 may be advanced per-orally again in this case and into the esophagus E, where an incision may be made through the esophageal wall. The overtube 14 may be temporarily attached to the esophageal wall and endoscope 50 then be advanced through this incision for accessing a region within the thoracic TC or peritoneal cavity PC.

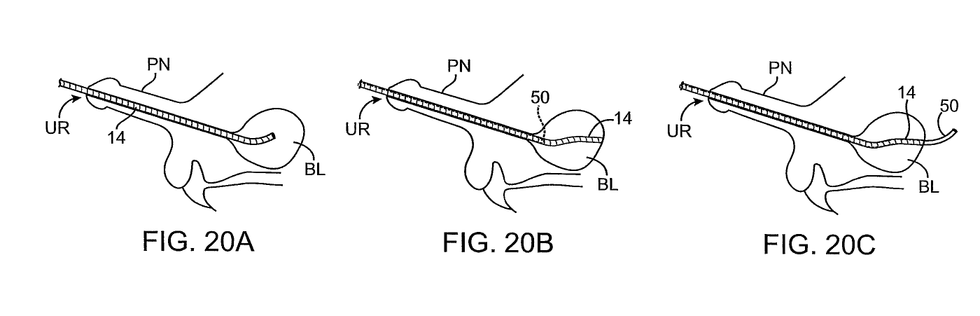

Yet another transluminal procedure is shown in FIGS. 20A to 20C. In this variation, an appropriately sized overtube 14 may be advanced through the urethra UR of a male patient's penis PN for access into the urinary bladder BL. Once access to the bladder interior is achieved, any number of procedures may be performed therewithin, as described above, or access into the peritoneal cavity PC may be achieved by passing the endoscope 50 or other instrument through the bladder wall.

In any of the transluminal procedures described above, the various methods and devices for maintaining sterility through the transluminal path may be utilized in combinations with one another and with any of the various procedures, as desired.

While illustrative examples are described above, it will be apparent to one skilled in the art that various changes and modifications may be made therein. Moreover, various tools or procedures described above are also intended to be utilized in combination with one another, as practicable. The appended claims are intended to cover all such changes and modifications that fall within the true spirit and scope of the invention.

What is claimed is:

1. A system for maintaining sterility of an elongate device advanced through a natural opening into a patient body, comprising:
an elongate device having a distal end, a proximal end, and a length therebetween, the elongate device being adapted for insertion into the patient body while in a flexible state; and
a sterile flexible sheath for placement over at least a portion of the elongate device inserted into the patient body, a distal end of the sheath being adapted to be opened when one or more instruments are advanced therethrough.

2. The system of claim 1 wherein the elongate device comprises a length adapted to transition between the flexible state and a rigid state.

3. The system of claim 1 wherein the flexible sheath is placed over at least a majority of a length of the elongate device.

4. The system of claim 1 wherein the flexible sheath is adapted to unroll over the elongate device.

5. The system of claim 1 wherein the distal end of the sheath is comprised of a thinned layer of material susceptible to rupture.

6. The system of claim 1 wherein the distal end of the sheath is perforated to facilitate rupturing.

7. The system of claim 1 wherein the sterile flexible sheath is adapted to retain the ruptured distal end in an open configuration upon withdrawal of the one or more instruments.

8. An apparatus for maintaining sterility of an interior of an elongate device advanced through a natural opening into a patient body, comprising:
a base member adapted to be secured at a distal end of the elongate device;
a membrane supported by the base member and adapted to cover an opening defined at the distal end of the elongate device, wherein the membrane is adapted to be opened upon being urged.

9. The apparatus of claim 6 wherein the base member comprises a circumferential cap or hood.

10. The apparatus of claim 6 wherein the elongate device comprises a distal end, a proximal end, and a length therebetween, the elongate device being adapted for insertion into the patient body while in a flexible state.

11. The apparatus of claim 8 wherein the length is adapted to transition between the flexible state and a rigid state.

12. The apparatus of claim 6 wherein the base member is secured to the distal end via a detent or projection.

13. The apparatus of claim 6 wherein the membrane comprises an elastic, plastic, or woven material.

14. An apparatus for maintaining sterility of an interior of an elongate device advanced through a natural opening into a patient body, comprising:
an elongate device having a distal end, a proximal end, and a length therebetween, the elongate device being adapted for insertion into the patient body while in a flexible state;
a flexible sheath attached near or at the distal end, wherein the flexible sheath is deployable from a low-profile configuration to an expanded configuration having a circumferential edge for contacting a tissue surface; and
at least one tissue engagement feature defined along the edge adapted to be engaged temporarily to the tissue surface.

15. The apparatus of claim 14 wherein the flexible sheath is configured to deploy via one or more struts.

16. The apparatus of claim 14 wherein the circumferential edge defines a lip or ledge defined radially outward relative to the flexible sheath.

17. The apparatus of claim 14 wherein the circumferential edge defines a lip or ledge defined radially inward relative to the flexible sheath.

18. The apparatus of claim 14 wherein the at least one tissue engagement feature is comprised of a plurality of openings or projections.

19. The apparatus of claim 14 further comprising at least one anchoring mechanism which is engageable with the tissue surface via the at least one tissue engagement feature, wherein the at least one anchoring mechanism is selected from the group consisting of clips, sutures, anchors, adhesive.

20. A method of maintaining sterility of an elongate device advanced into a patient body, comprising:
endoluminally advancing an elongate device into a patient body;
positioning the distal end of the elongate device proximate to a tissue surface to be opened;
deploying a flexible sheath attached near or at the distal end; and
securing a circumferential edge of the flexible sheath against the tissue surface.

21. The method of claim 20 wherein endoluminally advancing comprises advancing the elongate device per-orally into a stomach.

22. The method of claim 20 wherein positioning comprises steering the distal end of the elongate device into proximity to the tissue surface.

23. The method of claim 20 wherein securing comprises engaging the tissue surface with one or more projections defined along the circumferential edge.

24. The method of claim 20 wherein securing comprises engaging the tissue surface with an anchoring mechanism selected from the group consisting of clips, sutures, anchors, and adhesive.

25. The method of claim 20 further comprising flushing the tissue surface to be opened with a sterile fluid.

26. The method of claim 25 wherein flushing comprises flushing the tissue surface with a sterile fluid selected from the group consisting of water, saline, iodine, and other disinfecting solutions.

27. The method of claim 20 further comprising creating an opening through the tissue surface.

28. A method of maintaining sterility of an elongate device advanced into a patient body, comprising:
endoluminally advancing an elongate device into a patient body, wherein the elongate device is advanced while having a membrane disposed over an opening defined at a distal end of the elongate device;
positioning the distal end of the elongate device and membrane against a tissue surface to be opened; and
opening the membrane to allow access through the elongate device to the tissue surface.

29. The method of claim 28 wherein endoluminally advancing comprises inserting the elongate device trans-esophageally into a stomach.

30. The method of claim 28 wherein endoluminally advancing comprises inserting the elongate device trans-esophageally through an esophagus.

31. The method of claim 28 wherein endoluminally advancing comprises inserting the elongate device trans-anally into a colon.

32. The method of claim 28 wherein endoluminally advancing comprises inserting the elongate device trans-rectally into a peritoneal cavity.

33. The method of claim 28 wherein endoluminally advancing comprises inserting the elongate device trans-vaginally into a vagina.

34. The method of claim 28 wherein endoluminally advancing comprises inserting the elongate device trans-vaginally nito a peritoneal cavity.

35. The method of claim 28 wherein endoluminally advancing comprises inserting the elongate device trans-tracheally into a lung.

36. The method of claim 28 wherein endoluminally advancing comprises inserting the elongate device trans-urethrally into a bladder.

37. The method of claim 28 wherein positioning the distal end comprises steering the distal end into contact against the tissue surface.

38. The method of claim 28 further comprising temporarily attaching the distal end to the tissue surface.

39. The method of claim 28 wherein rupturing the membrane comprises advancing a piercing instrument through the membrane.

40. The method of claim 28 wherein rupturing the membrane comprises advancing an endoscope through the membrane.
